(19) United States
(12) Patent Application Publication
(10) Pub. No.: US 2024/0428404 A1
(43) Pub. Date: Dec. 26, 2024

(54) METHOD AND DEVICE FOR DETERMINING LIVER CONDITION OF SUBJECT

(71) Applicant: SUPERSONIC IMAGINE, Aix-En-Provence (FR)

(72) Inventors: William LAMBERT, Marseille (FR); Bo ZHANG, Simiane-Collongue (FR); Jonathan LAVAUD, Aix-en-Provence (FR)

(21) Appl. No.: 18/665,179

(22) Filed: May 15, 2024

(30) Foreign Application Priority Data
Jun. 16, 2023 (FR) 2306223

(51) Int. Cl. G06T 7/00

(52) U.S. Cl. CPC G06T 7/0012; G06T 2207/10132; G06T 2207/30056

(57) ABSTRACT

Examples of the disclosure relate to a method and a device for determining the condition of the liver of a subject. The method includes obtaining a first group of scan data from ultrasonic waves propagating according to a first scan configuration of the liver, obtaining a second group of scan data from ultrasonic waves propagating according to a second scan configuration of a group of tissues of the subject, and determining, by a combination of the first and second groups of scan data, an indicator representative of the condition of the liver.

### PRIOR ART

The present disclosure relates to the determination of the condition of the liver of a subject, such as a human or an animal, and covers in particular, yet not exclusively, assistance in screening or in diagnosing hepatic disorders such as non-alcoholic hepatic steatosis (NASH), also so-called non-alcoholic fatty liver disease (NAFLD), or non-alcoholic steatohepatitis (NASH).

Hepatic steatosis is a liver disorder which is characterised by the accumulation of fat in hepatic cells. There are many causes of hepatic steatosis, including in particular alcohol and the metabolic syndrome. Non-alcoholic hepatic steatosis, so-called NASH, affects persons who do not drink alcohol excessively. When more than 5% of the hepatocytes contain lipid droplets, we could then talk about pathological hepatic steatosis. Simple hepatic steatosis, so called steatosis, is generally benign and therefore does not cause considerable damages to the liver. On the other hand, this condition could evolve into more serious disorders, such as non-alcoholic steatohepatitis, so-called NASH, which corresponds to an aggressive affection characterised by the apparition of an inflammation within the hepatic parenchyma. Steatohepatitis itself could cause an accumulation of scar tissue (fibrosis) which could possibly lead to a potentially serious disease of the liver, namely cirrhosis.

Hepatic steatosis, in particular NASH, is nowadays considered as a major public health concern worldwide. About two million new cases of liver diseases are currently identified each year throughout the World. An increase by 21% and 63% respectively of the prevalence of NAFLD and of NASH between 2015 and 2030 is considered at this time. This hepatic condition is related to behavioural (physical activity, diet) and genetic factors. In particular, it affects overweight or obese persons. Studies have demonstrated that non-alcoholic hepatic steatosis increases the overall morbidity and the morbidity related to the cardiovascular condition, and could lead to various pathologies, in particular hepatic disorders, the formation of tumours, etc.

However, hepatic steatosis is a relatively slow and reversible process, which could, in particular, be treated effectively in particular through a better diet and more generally a better health practices, yet provided that care is taken early. Hence, it is critical to be able to screen and diagnose hepatic steatosis cases, in particular NASH, as early as possible. Yet, nowadays, there are different techniques for diagnosis, and more generally for monitoring the condition of the liver, these not always offering satisfactory results.

MRI examination is currently one of the reference non-invasive methods for evaluating the condition of the liver, and in particular for diagnosing and quantifying hepatic steatosis. In particular, it enables a uniform and non-invasive inspection of the entire liver. Thus, thanks to this technique, it is possible to determine an overall value of the fat ratio of a liver, this ratio being so-called PDFF standing for “Proton Density Fat Fraction”. However, this technique involves some constraints and limits, to the extent that it requires equipment and skills that are still not widely available (problem of cost, complexity of manufacture, scarcity, maintenance, training, etc.), and could even require a complex setting; which makes it barely suitable for some examinations of the liver, in particular for routine examinations, monitoring cases on a large scale, and, where possible, quite before the development of potential diseases, in order to make subjects profit from upstream treatments, since these pathologies are often potentially reversible, provided that they are detected early enough.

Hepatic biopsy is also a reference technique for this type of medical monitoring and diagnosis but has considerable risks and constraints, in particular because of its potential lack of reliability related to the intrinsically very local nature of this examination. Thus, this technique could lead to diagnosis errors, in particular if the biopsy has not targeted the liver in its most affected portion. It should be also noted that, because of its invasive nature, hepatic biopsy could lead to substantially severe complications, such as bleeding. Besides the problems of reliability and possible complication risks, biopsy has an expensive cost in financial terms and in terms of time invested by the subject and the medical staff.

Ultrasonic techniques have been proposed for the non-invasive diagnosis of hepatic steatosis, in particular for NASH at different stages thereof. Nonetheless, despite its efficiency, quite often demonstrated, this examination technique is sometimes poorly mastered nowadays and, consequently, it does not always offer satisfactory results in particular in terms of relevance of the acquired and processed data, accuracy and reliability. One limitation of this technique lies in particular in the strong dependence of the relevance of the results on the skills of the echographist practitioner. This could be explained in particular by the fact that this technique is based on an anatomical and qualitative approach, requiring the implementation of numerous parameters, which makes the appreciation of the results more subjective and therefore more difficult as it depends on the user. This dependence on the user becomes critical if the latter is not trained enough and/or does not have enough time to carry out the examination in good conditions.

### DESCRIPTION OF THE DISCLOSURE

One of the objects of the present invention is to address at least one of the problems or shortcomings described above.

In particular, it might be desirable to carry out an accurate and reliable determination (or evaluation) of the condition of the liver of a subject or patient, such as a human subject for example.

In particular, it might be desirable to evaluate the condition of the liver of a subject in order to screen early a dysfunction or its signs or diagnose a hepatic disorder such as hepatic steatosis, in particular non-alcoholic hepatic steatosis (or steatohepatitis) (NASH), or non-alcoholic steatohepatitis (NASH).

To this end, according to a first aspect, the present disclosure relates to a method for determining the condition of the liver of a subject, said method comprising:
obtaining a first group of scan data from ultrasonic waves propagating according to a first scan configuration of the liver;
obtaining a second group of scan data from ultrasonic waves propagating according to a second scan configuration of a group of tissues of the subject; and
determining, by a combination of the first and second groups of scan data, an indicator S3 representative of the condition of the liver.

The method according to the invention can include other features that can be taken separately or in combination, in particular among the embodiments that follow which are set out purely by way of example and can be combined or associated unless stated otherwise.

According to one example, the group of tissues of the subject corresponds to a portion of the subject other than the liver.

According to one example, the method is such that:
the first scan configuration is an intercostal scan configuration of the liver; and
the second scan configuration is a subcostal scan configuration of the liver.

According to one example, the indicator S3 comprises at least one from among:
an indicator defining an attenuation coefficient;
an indicator defining a speed of sound;
an indicator defining a backscattering coefficient;
an indicator of the elasticity of the liver;
an indicator defining a viscoelasticity of the liver; and
an indicator of propagation non-linearity.

According to one example, the method comprises:
comparing the indicator S3 with a reference value.

According to one example, the method comprises:
evaluating a first score (S1) from the first group of scan data; and
evaluating a second score (S2) from the second group of scan data; wherein the indicator S3 is determined from the first and second scores.

According to one example, the method is such that:
the first score is a first attenuation coefficient; and
the second score is a second attenuation coefficient;
wherein the indicator S3 defines a biomarker of a physiopathological condition of the liver.

According to one example, one amongst the first and second groups of scan data is obtained from a scan so-called anterior scan according to an anterior scan configuration and the other one is obtained from a scan so-called posterior scan carried out according to a posterior scan configuration, the anterior scan being carried out before the posterior scan;

wherein the anterior scan configuration is one amongst the first and second scan configurations and the posterior scan configuration is the other one amongst the first and second scan configurations.

According to one example, the configuration of the posterior scan is selected from among the first or second score obtained from the anterior scan.

According to one example, the indicator S3 is determined from an average of the first and second scores.

According to one example, the average is determined by a linear combination of the first and second scores S1, S2.

According to one example, the average is a least mean-square average.

According to one example, the average is determined by a non-linear combination of the first and second scores S1, S2.

According to one example, the determination of the indicator S3 comprises:
processing the first and second groups of data, or data obtained from the first and second groups of data, according to a processing model obtained using a neural network trained from training data and reference scores (or indicators), the training data comprising ultrasonic scan data or data obtained from the ultrasonic scan data.

According to one example, prior to the processing, the neural network is trained by machine learning during a training phase comprising:
obtaining the ultrasonic scan data representative of waves propagating according to the first and second scan configurations on test subjects;
determining the training data from the ultrasonic scan data;
obtaining, from MRI imagings carried out on the test subjects, the reference scores representative of the condition of the liver of the test subjects; and
determining, by comparison of the training data with the reference scores, the processing model used to estimate the indicator S3 from the combination of the first and second groups of scan data.

According to one example, the method comprises:
evaluating, from the indicator S3, a fat mass ratio of the liver of the subject.

According to a second aspect, the present disclosure may be implemented by (or involve) a computer program comprising instructions which, when the program is executed by a computer contributes to the implementation of the method according to the first aspect. In particular, the different steps of the method according to the first aspect may involve instructions of computer programs.

Such a computer program can use any programming language or equivalent, and it can be in the form of a source code, object code, or intermediate code between a source code and object code, such as in a partially compiled form, or in any other desirable form.

According to a third aspect, the present disclosure relates to a recording medium (or information medium), readable by a computer (or a processor), on which a computer program according to the second aspect of the present disclosure is recorded.

On the one hand, the recording medium can be any entity or device capable of storing the program, such as at least one volatile and/or non-volatile memory. For example, the medium can include a storage medium, such as a rewritable non-volatile memory, a ROM, a CD-ROM or a microelectronic circuit type ROM, or a magnetic recording medium or a hard disk. This memory can, for example, comprise a graphics card (or video card) memory, this type of memory being in particular designed to process image data (or video data).

On the other hand, this recording medium can also be a transmissible medium such as an electrical or optical signal, such a signal being able to be conveyed via an electrical or optical cable, via conventional or Hertzian radio or by self-directed laser beam or by other means. The computer program according to the present invention can in particular be downloaded using a wired or wireless network, local or otherwise (e.g. Bluetooth®, Wi-Fi, Ethernet, internet, 4G, 5G or others).

Alternatively, the recording medium can be an integrated circuit in which the computer program is incorporated, the integrated circuit being able to execute or be used in the execution of the method under discussion.

According to a fourth aspect, the present disclosure relates to a determination device (or monitoring device) configured to determine the condition of the liver of a subject by implementing the determination method of the first aspect of the present disclosure. To this end, the determination device may comprise modules configured to respectively implement the different steps of the method of the first aspect of the disclosure.

According to one example, the determination device comprises a memory associated with a processor, this memory comprising a computer program according to the second aspect of the disclosure.

According to one example, the device for determining the condition of the liver of a subject, this device comprising:
a first obtaining module configured to obtain a first group of scan data from ultrasonic waves propagating according to a first scan configuration of the liver;
a second obtaining module configured to obtain a second group of scan data from ultrasonic waves propagating according to a second scan configuration of a group of tissues of the subject; and
an analysis module configured to determine, by a combination of the first and second groups of scan data, an indicator S3 representative of the condition of the liver.

According to one embodiment, the invention is implemented by means of software and/or hardware components. In particular, the determination device may comprise modules configured to implement the steps of the method according to the first aspect of the disclosure. Thus, the different embodiments mentioned in this document in connection with the method according to the first aspect of the disclosure as well as the associated advantages could apply similarly to the determination device according to the fourth aspect of the present disclosure.

In this respect, the term “module” may correspond in the present disclosure to a software component, as well as a hardware component or a set of hardware and software components. A software component corresponds to one or more computer program(s), one or more subprogram(s) of a program, or more generally any element of a program or of a software able to implement a function or a set of functions, according to what is described hereinbelow for the considered module.

Similarly, a hardware component corresponds to any element of a hardware set able to implement a function or a set of functions, according to what is described hereinbelow for the considered module.

According to a fifth aspect, the present disclosure relates to a system for determining the condition of the liver of a subject, this system comprising:
a determination device according to the fourth aspect of the present disclosure; and
an ultrasonic system coupled to the determination device.

Advantageously, the determination method and device of the present disclosure allow determining, in a reliable, repeatable and effective manner, the condition of the liver of a subject, who could be healthy or have any physiopathological condition of the liver. To do so, the present disclosure is based on the combination of scan data (or ultrasonic data) obtained according to two distinct scan configurations, namely according to respectively a first scan configuration CF1 targeting the liver and a second scan configuration CF2 targeting the liver or another group of tissues of the subject. Advantageously, the combination of these scan data allows obtaining an indicator S3 representing the condition of the liver of the considered subject in a more reliable and robust manner than the case where only one scan configuration was used.

Hence, the determination method and device involve obtaining scan data according to different scan configurations. In the context of the present disclosure, a scan corresponds to an echographic (or ultrasonic) scan, an echographic (or ultrasonic) observation or a phase of observation by echography (or ultrasounds). Such a scan comprises the propagation of ultrasonic waves in a medium, namely an examined subject, and the recovery, in return, of echographic echoes (or ultrasonic echoes) representative of the condition of the liver of the subject. Each scan allows obtaining (or generating) scan data representative of one or more target region(s) (or area(s) of interest) of the subject.

The target region(s) of the subject that are examined (or scanned) by ultrasounds during an echographic scan depend on the used scan configuration. Various parameters may define this configuration, including in particular the orientation and/or the position of the emitter device during the echographic scan.

The combination of two distinct acquisition modes, for example in the intercostal mode and subcostal mode, advantageously allows improving the relevance of the acquisition data in comparison with a conventional technique where only ultrasonic data obtained in the intercostal mode would have been taken into account. The ultrasonic data thus acquired are more representative of the condition of the liver. Advantageously, using two distinct scan configurations allows diversifying the measurements and thus enhancing the reliability and the accuracy of the results.

Furthermore, in the case of hepatic steatosis for example, all the regions of the liver of the subject could be affected in different manners. Thus, some cases of heterogeneous steatoses have been noticed. The multiplication of ultrasonic scans according to different scan configurations allows obtaining a more representative estimation of the overall condition of the liver of the subject. Thus, the present disclosure may advantageously provide assistance in the diagnosis or in the monitoring of some physiopathological conditions of the liver, such as hepatic steatosis (for example of the NASH type), inflammatory steatosis, etc.

The features and advantages of the invention will become apparent more clearly upon reading the description below, provided purely by non-limiting way of example, and with reference to the appended figures. In particular, the examples illustrated in the figures could be combined together, provided that there are no mentioned or evident inconsistencies.

### BRIEF DESCRIPTION OF THE FIGURES

Other features and advantages of the present disclosure will appear from the description of the non-limiting embodiments of the present disclosure hereinafter, with reference to the appended FIGS. 1 to 16, wherein:

FIG. 1 schematically illustrates an intercostal scan configuration of an ultrasonic scan of the liver of the subject, according to a particular example;

FIG. 2 is an echographic image illustrating the apparition of shadow areas during the echographic examination of the liver of a subject in the intercostal acquisition mode;

FIG. 3 schematically shows a determination device and an ultrasonic system, according to an embodiment of the present disclosure;

FIG. 4 schematically shows an ultrasonic system, according to an embodiment of the present disclosure;

FIG. 5 schematically shows a determination device, according to an embodiment of the present disclosure;

FIG. 6 schematically shows scan configurations according to which ultrasonic scans are carried out, according to an embodiment of the present disclosure;

FIG. 7 schematically shows scan configurations according to which ultrasonic scans are carried out, according to an embodiment of the present disclosure;

FIG. 8 schematically shows a determination method carried out by the determination device of FIG. 3, according to an embodiment of the present disclosure;

FIG. 9 schematically shows results obtained in the intercostal acquisition mode, according to an embodiment of the present disclosure;

FIG. 10 schematically shows results obtained in the subcostal acquisition mode, according to an embodiment of the present disclosure;

FIG. 11 schematically shows results obtained according to the determination method by combining the results of FIGS. 9 and 10, according to an embodiment of the present disclosure;

FIG. 12 schematically shows the distribution of the PDFF scores of the liver of test subjects during a study validating the efficiency of the determination method, according to an embodiment of the present disclosure;

FIG. 13 schematically shows a determination method, according to an embodiment of the present disclosure;

FIG. 14 schematically shows a determination method, according to an embodiment of the present disclosure;

FIG. 15 schematically shows a neural network implemented by the determination device of FIG. 3, according to an embodiment of the present disclosure; and

FIG. 16 schematically shows a training phase carried out by the neural network of FIG. 15, according to an embodiment of the present disclosure.

### DESCRIPTION OF THE EMBODIMENTS

The present disclosure covers, in particular, devices and methods for determining the condition of the liver of a subject, for example for a screening campaign, medical monitoring or diagnosis assistance.

It is known to carry out the examination of the liver of a subject by echography, in particular in order to screen or diagnose hepatic disorders such as hepatic steatosis (of the NAFLD type for example) or NASH-type steatohepatitis. Resorting to this technique is advantageous in particular because of its non-invasive nature, the relative availability on the field of echographic apparatuses for medical examinations, the ease of moving them as the need might be, and the lower costs and risks of the investments and of the examinations, for example, in comparison with MRI and biopsy.

Nonetheless, this examination technique, the relevance of the results thereof is considerably conditioned by the level of qualification of the practitioner, is still at its beginnings and does not always offer satisfactory results, in particular in terms of performances and reliability. These problems may have several origins as discussed hereinafter.

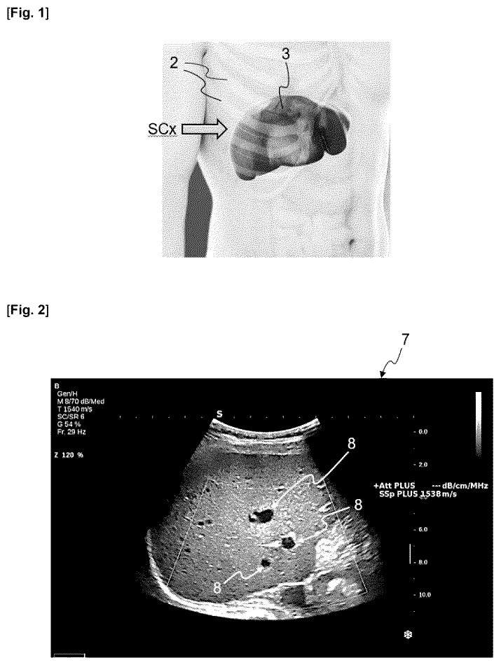

As illustrated in FIG. 1, the practice that is widely established worldwide currently consists in carrying out such an echographic examination of the liver according to a so-called “intercostal” scan configuration, denoted SCx (FIG. 1), i.e. by positioning the ultrasonic probe so that the ultrasonic waves propagate between the ribs 2 of the subject in order to recover, in return, an ultrasonic echo representative of the liver 3 of the subject. To do so, the ultrasonic probe, possibly coated with a suitable gel, is positioned in contact with the skin of the subject at the level of his/her thoracic cage, so as to scan “throughout the ribs”, i.e. between the ribs.

This intercostal scan configuration is considered as the most reliable, in particular because of the fact that the ribs of the subject protect the underlying tissues and therefore allow limiting the deformations that the probe might cause when it is applied in contact with the patient. Indeed, such deformations might distort the results of the observations, the compressed tissues then behaving differently. The intercostal configuration is also preferred to the extent that this facilitates the examination, in particular for some subjects difficult to examine (in particular those having a high subcutaneous fat level). The echographist technician may consider the ribs of the subject as a reference to best orient and position the probe of the echography system.

In particular for the aforementioned reasons, the clinical studies currently recommend adopting the intercostal scan configuration to carry out an examination of the liver, in particular in order to screen or diagnose hepatic disorders such as the aforementioned ones.

However, it has been noticed that the echographic examination of the liver according to the intercostal technique poses some problems and implies limits that should be addressed. As illustrated as example in FIG. 2, it has been observed in particular the apparition of shadow areas 8 in the echographic images 7 obtained according to the intercostal scan configuration. These dark areas 8, which hinder the observation, are explained by the generally convex shape of the ultrasonic probe which does not always enable the latter to be completely in contact with the skin of the subject during the examination, in particular because of the ribs of the subject which form reliefs that are barely compatible with the shape of the probe. The non-contact areas between the probe and the skin of the subject during the echographic observation could hinder the propagation of the ultrasonic waves and lead to the formation of these areas which then appear dark 8 in the image.

Nonetheless, it is difficult, and even not desirable, to modify the shape of the probe to better match with the profile of the ribs, in particular because the current configurations are designed so as to maximise the field of view of the echographic system and ensure some versatility of the probe, which enables the latter to be adapted to carry out various echographic examinations.

Besides the aforementioned problems of shadow areas, the echographic observation of the liver according to the conventional intercostal scan configuration generally suffers from results that are sometimes unsatisfactory. It has been noticed that the evaluation of some characteristics of the liver, such as some biomarkers, is not always effective or reliable enough in the case of such uses.

Hence, the present disclosure aims to provide a solution in particular to the aforementioned problems and shortcomings, allowing determining with more reliability and better performances the condition of the liver of a subject, such as a human being or an animal, or more generally the condition of the liver of a living being for example, in particular, yet not exclusively, to assist in screening or in diagnosing hepatic disorders, or more generally to evaluate the condition of the liver of the subject. In particular, this solution is based on a method and a device for determining the condition of the liver of a subject.

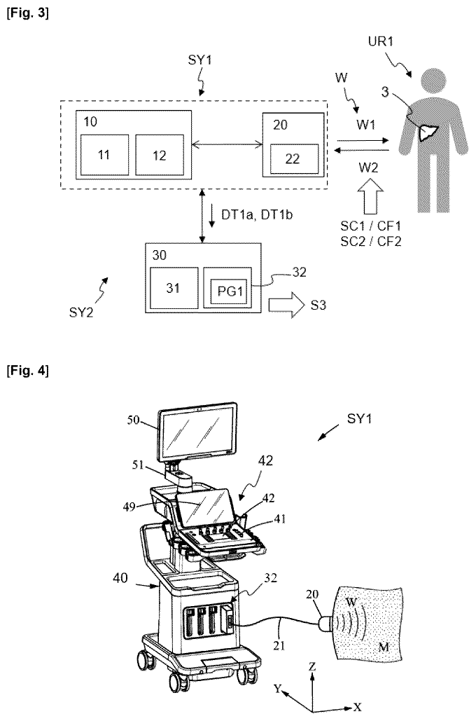

Determination methods and determination devices (also so-called “methods” and “devices”) will be described hereinafter according to particular embodiments of the disclosure with reference to FIGS. 3 to 16 together. Unless stated otherwise, common or similar elements in several figures bear the same reference numerals and have identical or similar features such that these common elements are not generally described again for concision.

The terms “first” (or “first, second”, etc.) are used in this document by arbitrary convention to enable different elements (such as operations, devices, etc.) carried out or used in the embodiments described below to be identified or distinguished.

As illustrated in FIG. 3, embodiments of the present disclosure are described hereinafter as examples wherein the liver 3 of a subject UR1 is observed by echography by means of an ultrasonic system denoted SY1. To do so, ultrasonic waves W are emitted in different regions (or areas of interest) of the subject UR1 by means of the ultrasonic system SY1 in order to obtain ultrasonic data representative of the condition of the liver 3. This observation comprises several echographic scans, i.e. several phases during which ultrasonic waves propagate and ultrasonic echoes, representative of the condition of the liver 3, are recovered, in return. In other words, during each scan, one or more region(s) of the liver are insonified by incident ultrasonic waves and echoes backscattered by these regions of the liver are captured, in return, by means of an ultrasonic probe. Next, we will interchangeably use the terms “scan”, “echographic (or ultrasonic) scan”, “echographic (or ultrasonic) observation”, “phase of observation by echography (or ultrasounds)” or “acquisition phase”.

In the considered examples, the subject is a living being, namely a mammal, for example a human being. Nonetheless, it should be noted that the concept of the present disclosure could apply more generally to any subject having a liver, (human or animal), living or dead, the condition of the liver of whom is to be evaluated.

It should be noted that the subject UR1 used to illustrate the present disclosure may have a body in any physiological condition, in particular at the hepatic level. In particular, the liver 3 of the subject may be healthy (for example no particular hepatic disorder) or suffer from any hepatic disorder or pathology.

The method and device of the present disclosure are based, in particular, on obtaining two groups of data, respectively from ultrasonic waves W propagating during ultrasonic scans, of the same subject, according to different scan configurations, at least one of these configurations being a scan configuration of the liver, and on the combination of these two groups of data to determine an indicator denoted S3 (also so-called overall indicator, or overall score, or parameter) which is representative of the condition of the liver 3 of the subject UR1. Advantageously, the combination of these two groups of data allows determining an indicator representative of the condition of the liver of the subject.

In the present disclosure, a scan configuration of the liver refers to a scan configuration according to which echographic data representative of the liver of the subject are obtained. In other words, an echographic scan carried out according to a scan configuration of the liver scrutinises (or aims at, or targets), at least in part, the liver of the subject, so as to obtain echographic data representative of said liver.

According to some embodiments, the determination method comprises:
obtaining a first group of scan data from ultrasonic waves propagating according to a first scan configuration of the liver;
obtaining a second group of scan data from ultrasonic waves propagating according to a second scan configuration of a group of tissues of the subject; and
determining, by a combination of the first and second groups of scan data, an indicator S3 representative of the condition of the liver

As illustrated as example in FIG. 3, different echographic scans (or observations) are considered hereinafter, namely a first scan SC1 and a second scan SC2, which are carried out by means of the ultrasonic system SY1 on a subject UR1 according to different scan configurations (or modes)—respectively denoted CF1 and CF2—in order to determine the condition of the liver 3 of the subject UR1. The scan configurations CF1 and CF2 may vary as the case might be, in particular in terms of scan direction and/or position with respect to the subject, yet to the extent that these configurations are slightly or more generally different from one another.

A determination device (also so-called processing device) 30 is further configured to obtain and process scan data (or ultrasonic data) DT1a and DT1b generated by the ultrasonic system SY1 respectively from the first and second scans SC1 and SC2 (FIG. 3). To do so, the determination device 30 cooperates (is coupled) with the ultrasonic system SY1 to implement the determination method according to at least one particular embodiment.

As illustrated in FIG. 3, the determination device 30 and the ultrasonic system SY1 together form a determination system (or analysis system) SY2, this system being configured to determine the condition of the liver of a subject.

According to one example, the determination device 30 is an integral part of the ultrasonic system SY1. According to one variant, the determination device 30 is external to (distinct from) the ultrasonic system SY1.

The system SY1 can be a medical ultrasound system. Consequently, the emitter device 20 may be (or be part, at least partially, of) a medical and/or ultrasonic probe.

Embodiments of the ultrasonic system SY1 are now described for merely illustrative purposes.

FIG. 3 schematically shows an embodiment of the ultrasonic system SY1 which comprises a control device (or steering device) 10 and an emitter device 20. The control device 10 is configured to steer the emitter device 20. The emitter device 20 is configured to emit and receive ultrasonic waves W, towards and from a studied medium, namely the subject UR1 in the considered examples, and that being so under the control of the control device 10.

According to a particular example, all or part of the determination device 30 is part of the control device 10. According to one example, the devices 10 and 30 form the same device.

For example, the emitter device 20 is an ultrasonic transducer device (or ultrasonic probe) configured to emit and/or receive ultrasonic waves W. For example, this emitter device 20 is included in an ultrasonic probe intended to be applied in contact with the subject UR1 during echographic scans to determine the condition of his/her liver 3.

It should be noted that various configurations of the emitter device 20 are nonetheless possible. In particular, all or part of the control device 10 may be implemented in the emitter device 20 (for example in the probe). All or part of the control device 10 may be external to the emitter device 20 (or to the probe).

The control system 10 may comprise in this example a processing unit (or module) 11 and a control unit (or module) 12. The control unit 12 (also so-called power control unit) is configured to steer the emitter device 20 by means of electrical signals which are exchanged or transmitted) between the system 10 and the transducer device 20. Thus, the control unit 12 generates electrical signals which are transmitted to the transducer device 20 to cause the emission, by said transducer device 20, ultrasonic waves W1 in the direction of the area of interest, located in the subject UR1. The electrical signals thus generated are representative of (or defining) the ultrasonic waves projected in the medium. To this end, the control unit 12 may, for example, consist of or comprise at least one electronic pulser, also so-called “pulser”.

The control unit 12 may possibly comprise receiver devices or receiver circuits (not shown) configured to receive electrical signals originating from the transducer device 20.

The processing unit 11 may be configured to control the control unit 12, for example by controlling the electrical signals. For example, the processing unit 11 may consist of or comprise at least one processor.

According to one example, the processing unit 11 and/or the steering unit 12 are included in the body 31 (central element) of the system SY1 shown for illustration in FIG. 4.

More particularly, the processing unit 11 may be configured to control the electrical signals that are generated by the control unit 12. The processing unit 11 may also be configured to process (and/or interpret) electrical signals possibly received by the control unit 12 originating from the emitter device 20. These signals are representative of waves W2 received by the transducer device 20 originating from the subject UR1 (FIG. 3). For example, these waves W2 form one or more ultrasonic echo(s), i.e. a response of the studied medium (namely the liver of the subject UR1) to the ultrasonic waves emitted in the direction of said medium. The processing carried out by the processing unit 11 on the received signals may vary depending on the case and comprise for example at least any one from among signal amplification, filtering, digitisation and conditioning operations.

In this disclosure, the term “medium” refers to one or more area(s) of interest (or region(s)) of the subject UR1, i.e. the areas that are insonified or scrutinised by ultrasounds, namely areas of interest of the liver 3 and possibly areas of interest corresponding to other portions of the subject UR1.

As illustrated in FIG. 3, the emitter device 20 may comprise, for example, one or more ultrasonic transducer(s) (also so-called transducer elements) 22, each being configured to convert an electrical signal received from the control device 10 into ultrasonic waves (and possibly also reciprocally). Thus, the transducers 22 may be configured to emit waves (or ultrasonic impulses, or ultrasonic beams) W1 in the medium, which corresponds to an emission operation.

The transducers 22 may also possibly be configured to receive, during a reception operation, ultrasonic signals W2 originating from one or more area(s) of interest of the subject UR1, for example in response to the transmitted waves W1. These received signals W2 may be in the form of a set of echo signals backscattered in response to the signals W1 emitted beforehand. For example, each transducer 22 may convert a received echo signal W2 into an electrical signal. Afterwards, the signal may be processed by the control device 10 (for example by the processing unit 11) or by any associated (dedicated) system, directly connected or not. For example, the signal W2 may be amplified, filtered, digitised and/or an operation of conditioning the signal may be performed. The transducer elements may be arranged according to a row of transducers, an array, or as a network of transducers or any other configuration.

It should be noted that the same transducer(s) 22 may be used to emit waves (or impulses) W1 and, where appropriate, receive the waves W2 forming the response of the medium, or different transducers may be used for the emission and the reception of the waves. There could be one or more emitter transducer(s), and possibly a plurality of receiver transducers. In another variant, the same transducer 22 may be used for the emission and the reception of the ultrasonic waves. The transducers 22 may comprise piezoelectric crystals and/or other components which may be configured to generate and/or record and/or receive signals.

The control device 10 may comprise the emitter device 20. Alternatively, the emitter device 20 may be external to the control device 10. For example, the emitter device 20 may be connected to the control device 10 by a cable or may communicate wirelessly with it. In the latter case, the emitter device 10 may, for example, comprise a battery and receive communication signals from the control device 10 which represent the electrical signals (for example the steering frequencies and/or any information included in the electrical signals). The emitter device 20 could then replicate the electrical signals internally from the received communication signals.

For example, the emitter device 20 may be a conventional ultrasonic emitter device. Thus, a difference according to the present disclosure might lie in the manner in which the ultrasonic data DT1a and DT1b are obtained and processed by the determination device 30 to determine the condition of the liver 3 of the subject UR1, as described in more details hereinafter.

The control device 10 may be stationary or movable. The emitter device 20 may also be stationary or movable. For example, the control device 10 may be a fixed system (for example comprising a processing unit and a display device, as described hereinbelow) and the emitter device 20 may be movable (for example a sensor device, a measurement device, or a probe). Nonetheless, it is also possible that the emitter device 20 is integrated into the control device 10, and that the device 10 is a movable system. For example, the control device 10 may be configured to be steered in a standalone manner, for example thanks to an embedded battery. Other examples are described hereinbelow.

According to one example, the control device 10 may comprise at least one memory (not shown) used by the processing unit 11 to control the control unit 12. This memory may possibly be part of the processing unit 11. In some examples, the processor and the memory of the processing unit 11 may be incorporated into the control device 10 illustrated in FIG. 3 or may be incorporated into a computer or a computer device connected so as to communicate with the latter.

Depending on the configuration and type of the considered computer device, the memory of the control device 10 may be volatile (such as the RAM), non-volatile (such as the ROM memory, flash, EEPROM, etc. or any other storage device and/or computer-readable medium as described hereinafter) or a combination of both. For example, this memory may be generated in the DMA (standing for “Direct Memory Access” in English) mode. For example, the memory used by the processing unit 11 may comprise all or part of a graphical card (or video card) memory, this type of memory being in particular able to process and/or send image data that could be used to display one or more image(s) on a display screen (or unit).

More generally, the control device 10 may comprise (removable and/or non-removable) storage devices, including, yet without limitation, magnetic or optical disks or tapes.

Furthermore, the control device 10 may include one or more input device(s) such as a keyboard, a mouse, a stylet, a voice input, etc. and/or one or more output device(s) such as one or more screen(s), loudspeakers, a printer, etc. The environment may also comprise one or more communication connection(s), such as LAN, WAN, point-to-point type connections, etc. In some embodiments, the connections may be used to establish point-to-point communications, wired communications, wireless communications, etc.

The control device 10 may be a unique computer operating in a networked environment using logical connections with one or more remote computer(s). The remote computer may be a review station, a personal computer, a server, a router, a network PC, a similar device or another common network node, and may generally comprise several ones or all of the above-described elements as well as others that are not mentioned. The logical connections may include any method supported by the available communication media.

As already indicated, the embodiments of the ultrasonic system SY1 are set out hereinabove for merely illustrative purposes, other implementations being possible in the context of the present disclosure.

Moreover, the determination device 30 comprises processing means for obtaining and processing the scan data (or ultrasonic data) DT1a and DT1b generated by the ultrasonic system SY1 (FIG. 3). The scan data generated by the system SY1 may be analysed by the determination device 30 either in real-time, for example by means of an artificial intelligence algorithm and/or module, or analysed later on and/or in a location other than that where the ultrasonic system SY1 is located.

As described in more details hereinafter, the nature of the scan data DT1a and DT1b provided by the echographic system SY1 to the determination device 30 may vary as the case might be. For example, these data may comprise image data (or video data) and/or other data obtained from the scan data or the scan data themselves.

More specifically, in the example shown in FIG. 3, the determination device 30 comprises at least one processor 31 and at least one computer-readable medium such as a non-volatile memory 32. This memory 32, rewritable or of the ROM type, forms a recording medium (or information medium) in accordance with a particular embodiment, readable by the processor 31, and on which a computer program PG1 in accordance with a particular embodiment is recorded. This computer program PG1 includes instructions for the execution of the steps of a determination method according to at least one particular embodiment. In particular, this program may define instructions for processing ultrasonic data and/or instructions for constructing illustrative images of the observed medium.

For example, the computer program PG1 may comprise at least one artificial intelligence (AI) algorithm that could be executed by the processor 31 to implement the determination method according to a particular embodiment of the present disclosure. In particular, the computer program PG1 may define an artificial intelligence model (or predictive model) trained by means of training data to process the ultrasonic data obtained according to the determination method of the present disclosure, in particular to determine one or more indicator(s) (or score(s)) as described hereinafter in some embodiments.

The computer-readable media of the determination device 30, such as the memory 32, may consist of any available medium to which the processor 31 or its operating environment could access. For example, and without limitation, the computer-readable media may comprise computer storage media and communication media. The computer storage media comprise volatile and non-volatile media, removable and non-removable, implemented in any information storage method or technology such as computer-readable instructions, data structures, program modules or other data. The computer storage media do not include the communication media.

According to one example, the determination device 30 is configured to receive scan data, such as the data DT1a and DT1b, provided by the ultrasonic system SY1, process these data, and possibly send a result of this processing to an external device, like for example, a storage, display device, a server, or any other external device.

The communication between the determination device 30 and the ultrasonic system SY1 may be performed according to any communication link, wired or wireless as the case might be.

FIG. 4 schematically shows an embodiment of the ultrasonic system SY1 as described before with reference to FIG. 3, namely an ultrasonic imaging system in the considered examples.

Thus, the ultrasonic imaging system 10 as shown in FIG. 4 comprises:
the emitter device 20 in the form of an ultrasonic probe,
a processing unit 40 configured to process or generate an image on the basis of the ultrasonic signals received from the probe 20 (this unit 40 corresponding for example to the processing unit 11 of FIG. 3),
a control panel 42 associated with, linked or connected to the processing unit 40, said control panel possibly comprising at least one from among buttons 41 and a tactile tablet 42, and
one or more screen(s) 50 configured to display the image(s) generated by the processing unit 40.

The probe 20 may be connected to the processing unit 40 by any suitable connection means such as a cable 21 or a wireless connection. The probe 20 is also capable of emitting ultrasonic waves W in a medium M, and also possibly receiving ultrasonic waves W from the medium M, said received ultrasonic waves could then be substantial or resulting from reflections of said emitted ultrasonic waves on scattering particles inside said medium.

The processing unit 40 may comprise receiver devices (not shown), comprising (or being) for example receiver circuits, configured to process (for example amplify and/or filter) electrical signals received from the probe 20. For example, these receiver devices may comprise converters to transform the received signals into data representing the signal (for example analog-to-digital converters (ADC) configured to transform a voltage into a digital code). The obtained ultrasonic data may be processed in various manners; in particular, they may be temporarily stored in a memory accessible to the processing unit or processed directly to calculate intermediate processed data (data derived from a route formation process, so-called “beamformed data” in English). The processing unit 30 may implement any known processing method to generate and/or process one or more image(s) or map(s) or data on the basis of the signals received from the probe, such as the formation of routes.

The processed data may be associated with an ultrasonic image of different natures, which may be:
a B-mode image of the medium (B-mode image) generally displayed in shades of grey allowing visualising the organs inside the medium, and/or
a so-called Doppler image, showing the movements of fluids in the observed medium, for example the speed of movement and/or the flow of fluids in the medium (image often using colour codes), for example useful to visualise the blood vessels and their activities in the medium, and/or
an image showing a mechanical characteristic of the medium (elasticity), for example useful to identify areas of different hardnesses which might turn out to be lesions present inside the medium (for example shear wave elastographic image data (ShearWave™M Elastography)).

The display screen 50 (for example mounted on a support arm 51) may be a screen for displaying the image processed by the processing unit 30 and/or may display various pieces of information depending on the use case, in particular assistance information or contextual gesture assistance adapted or adaptable to the touchpad 42.

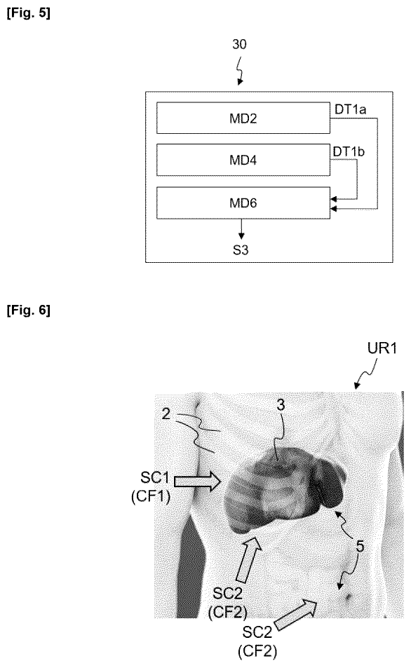

As schematically illustrated in FIG. 5 according to one example, the processor 31 of the determination method 30, steered by the computer program PG1 (FIG. 3), implements a given number of modules, namely: a first obtaining module MD2, a second obtaining module MD4 and a determination module (or analysis module) MD6.

More specifically, the first obtaining module MD2 is configured to obtain a first group of scan data DT1a from ultrasonic waves W1 propagating according to a first scan configuration CF1 of the liver 3 (FIG. 3). These first scan data DT1a, obtained during a first ultrasonic scan SC1, are representative of the liver 3 of the subject UR1.

The second obtaining module MD4 is configured to obtain a second group of scan data DT1b from ultrasonic waves W1 propagating according to a second scan configuration CF2 of a group of tissues of the subject 3 (FIG. 3). As described later on, these second scan data DT1a, obtained during a second ultrasonic scan SC2, are representative of any group of tissues, which may comprise all or part of the liver 3 of the subject UR1 or a portion (or region) of the subject UR1 other than his/her liver 3 (for example an organ of the abdomen other than the liver, such as the pancreas and/or the gallbladder).

The determination module MD6 is configured to determine, by a combination of the first and second groups of scan data DT1a and DT1b, an indicator (or overall indicator) S3 representative of the condition of the liver 3 of the subject UR1. As described later on in some examples, the processing applied to the data DT1a and DT1b to obtain the indicator S3 may be of various kinds. Similarly, the nature of the indicator S3 may vary as the case might be.

Embodiments of the present disclosure are now described with reference to FIGS. 6-12. In these examples, the previously-described determination device 30 implements the determination method according to some embodiments by executing the program PG1 (FIG. 3). To do so, the determination device 30 cooperates with the ultrasonic system SY1.

For example, it is assumed that it is desired to determine the condition of the liver 3 of a subject UR1 (FIGS. 6-8). To do so, a practitioner, physician, qualified technician, echographist, or more generally a user, carries out, by means of the ultrasonic system SY1 (FIG. 3), two ultrasonic scans on the subject UR1, namely:
a first ultrasonic scan SC1 during which ultrasonic waves propagate according to a first scan configuration CF1 of the liver 3; and
a second ultrasonic scan SC2 during which ultrasonic waves propagate according to a second scan configuration CF2 of tissues 5 of the subject UR1.

During each ultrasonic scan SC1 and SC2, the ultrasonic system SY1 emits ultrasonic waves W1 towards the targeted medium (the portion of the subject UR1 being observed) and receives, in return, ultrasonic echoes in the form of waves W2 (FIG. 3). These ultrasonic waves W2 are representative of the region of the subject UR1 in which the emitted waves W1 and the received waves W2 propagate. Thus, the ultrasonic system SY1 (and more particularly, the control device 10) generates ultrasonic data, so-called first and second scan data DT1a and DT1b, respectively from the ultrasonic echoes captured during the ultrasonic scans SC1 and SC2.

The region(s) of the subject UR1 which are subjected to observation by ultrasounds, as well as the ultrasonic echoes W2 obtained during this observation, depend on the configuration of the completed ultrasonic scans SC1 and SC2. Various parameters could define these configurations, including in particular the orientation and/or the position of the emitter device 20 during the ultrasonic scans.

For example, it is assumed that the first ultrasonic scan SC1 is configured to scan (scrutinise) all or part of the liver 3 of the subject UR1. In other words, the scan configuration CF1 is such that the ultrasonic waves propagating during the first scan SC1 are representative of an area of interest (or region) of the liver 3. Hence, the first ultrasonic scan SC1 is a scan of one or more area(s) of interest of the liver.

Furthermore, it is assumed for example that the second ultrasonic scan SC2 is configured to target (or scrutinise) a group of tissues 5 of the subject UR1 (FIG. 6), the nature of these tissues could vary as the case might be. In other words, the scan configuration CF2 is such that the ultrasonic waves propagating during the second scan SC2 are representative of the group of tissues 5.

According to one example, the group of tissues 5 scrutinised during the second scan SC2 comprises all or part of the liver (scan of the liver). Thus, the scan configurations (or modes) CF1 and CF2 are scan configurations of the liver.

According to one example, the scrutinised group of tissues 5 comprises (or corresponds to) a portion (or region) of the subject UR1 other than his/her liver 3, such as for example all or part of his/her gallbladder, an organ of the abdomen and/or the visceral fat of the subject UR1.

As described hereinafter, taking account of the information regarding the condition of a region other than the liver 3 of the subject could assist in the analysis and in the understanding of the condition of the liver 3, in particular because the condition of the liver 3 and that of other regions of the subject could be correlated.

For example, the ultrasonic observation of the visceral fat of the subject UR1 could provide useful information to determine the condition of the liver 3, in particular in order to assist in screening or diagnosing a hepatic disorder, because the visceral fat could form a biomarker of a pathological condition of the liver. For example, an excessive presence of visceral fat may be symptomatic of hepatic steatosis (for example of the NASH type), an intoxication of the liver, or another condition of the liver.

The group of tissues 5 may be selected according to the considered examination type, and/or assumed condition of the liver that we wish to study or confirm or the evolutions of which over time or with regards to a treatment we wish to study, for example during a medical examination. Irrespective of the region 5 of the subject that is scrutinised during the second ultrasonic scan SC2, the first and second scan configurations CF1 and CF2 are different in order to obtain ultrasonic data carrying different and complementary information.

As schematically shown in FIG. 7, it is assumed hereinafter for merely illustrative purposes that the ultrasonic scans SC1 and SC2 are configured so that:
the first scan configuration CF1 according to which the first ultrasonic scan SC1 is carried is of the intercostal type, i.e. by propagating ultrasonic waves throughout (or between) the ribs of the subject UR1 (positions P1); and
the second scan configuration CF2 according to which the second scan SC2 is of the subcostal type, i.e. by propagating ultrasonic waves under the thoracic cage throughout the abdomen (positions P2).

Thus, by positioning the ultrasonic probe 20 on the abdomen of the subject, it is possible to observe the liver of the subject according to another section, and thus improve the relevance of the obtained results.

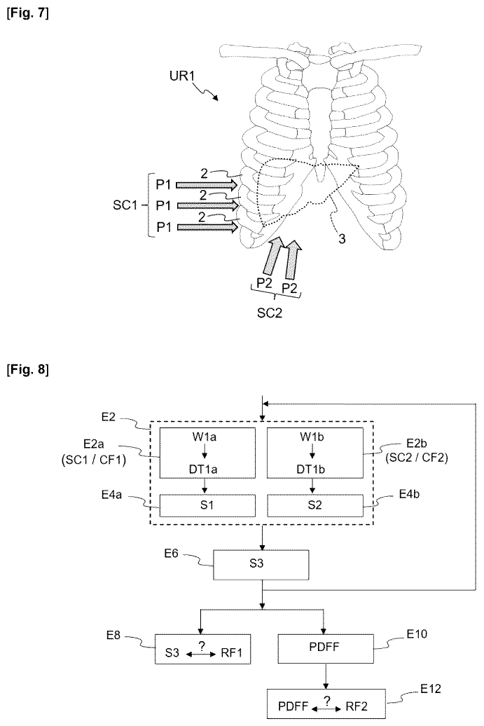

Thus, during a step E2a (FIG. 8), the determination device 30 obtains a first group of scan data DT1a from ultrasonic waves propagating during the first ultrasonic scan SC1 according to the first scan configuration CF1 of the liver 3.

Similarly, during a step E2b (FIG. 8), the determination device 30 obtains a second group of scan data DT1b from ultrasonic waves propagating during the second ultrasonic scan SC2 according to the second scan configuration CF2 of the group of tissues 5.

To do so, the ultrasonic system SY1 (and more particularly the control device 10) generates the scan data DT1a and DT1b from the ultrasonic echoes received during respectively the first and second ultrasonic scans SC1 and SC2. These scan data DT1a and DT1b are transmitted to the processing device 30 for processing according to the determination method.

Each ultrasonic scan SC1 and SC2 may comprise one or more scan(s) in the corresponding scan configuration CF1 and CF2, respectively.

The obtaining steps E2a and E2b are included in an obtaining phase denoted E2 (FIG. 8). Nonetheless, it should be noted that various arrangements of steps E2a and 2b are possible over time. Steps E2a and E2b may be carried out either simultaneously (at least in part), or one after another, either immediately after, or later on (for example during different examination sessions). According to one example, the first group of scan data DT1a is received before the second group of scan data DT1b (or vice versa).

Furthermore, the determination device 30 may receive the scan data DT1a and DT1b in real-time, while carrying out the scans SC1 and SC2, or subsequently, in a grouped manner or separately.

During a determination step E6 (FIG. 8), the determination device 30 determines, by combination of the first and second groups of scan data DT1a and DT1b, an indicator (or score) S3 representative of the condition of the liver 3 of the subject UR1. This combination may comprise a processing taking account of the data DT1a and DT1b, the nature of this processing could be adapted as the case might be as described hereinafter in some examples.

Steps E2-E6 may be repeated iteratively to improve or update the indicator S3 determined in E6.

According to one example, the indicator S3 defines a (or at least one) biomarker of a physiopathological condition of the liver. For example, the indicator S3 could inform on a hepatic disorder affecting the liver 3 of the subject UR1, for example a hepatic steatosis (of the NASH type for example), a steatohepatitis, a hepatic intoxication, etc.

According to one example, the indicator S3 determined in E6 (FIG. 8) comprises an attenuation coefficient which represents an attenuation of the ultrasonic waves during their propagation in the areas of interest of the subject UR1 during the first and second ultrasonic scans SC1 and SC2, respectively.

According to one example, the indicator S3 determined in E6 (FIG. 8) comprises an indicator defining, for example, a fat mass percentage (or ratio) of the liver. This percentage informs on the average amount of fat present in the liver 3 of the subject UR1.

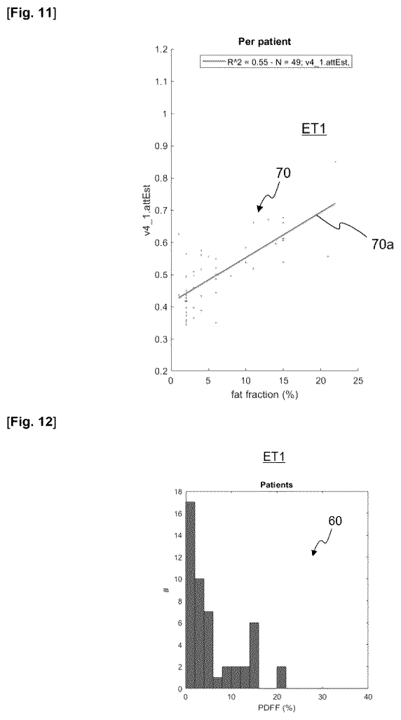

FIG. 11 shows, in the form of a cloud of points 70, the indicators S3 determined (E6) for a group of test subjects during a study ET1 carried out by the Applicant. The indicators S3 define a fat mass ratio, or PDFF, determined respectively for each subject of the study ET1.

FIG. 12 shows, for reference, the PDFF values determined for the identical subjects of the study ET1 by a technique other than that of the determination method, namely by MRI imaging. The evaluation of the PDFF by MRI imaging is currently the reference for predicting or diagnosing a hepatic steatosis. It should be noted that this study ET1 covers, in this case, subjects having a PDFF lower than a limit value, namely 25%.

According to one example, the indicator S3 determined in E6 (FIG. 8) comprises an indicator defining a speed of sound, i.e. the speed of the ultrasonic waves propagating in the liver of the subject UR1, or more specifically in the insonified area(s), during the ultrasonic scans SC1 and SC2.

According to one example, the indicator S3 determined in E6 (FIG. 8) comprises an indicator defining a backscattering coefficient (also so-called “backscattering coefficient”), i.e. a coefficient representative of a level of backscattering caused by the propagation of the ultrasonic waves in the medium during the ultrasonic scans SC1 and SC2. This backscattering coefficient is one amongst the physical parameters related to the capacity of the probed media to generate an echo propagating towards the ultrasonic probe.

According to one example, the indicator S3 determined in E6 (FIG. 8) comprises a liver elasticity indicator (or score), i.e. an indicator representative of the capacity of the tissues of the liver to deform in response to a mechanical stress to which they are subjected.

According to one example, the indicator S3 determined in E6 (FIG. 8) comprises an indicator (or score) defining a viscoelasticity of the liver, i.e. a coefficient representative of the viscosity and elasticity capacities of the liver.

According to one example, the indicator S3 determined in E6 (FIG. 8) comprises a scattering non-linearity indicator (or score), i.e. an indicator representative of the propagation non-linearity of the ultrasonic waves in the medium (in the liver) during the ultrasonic scans SC1 and SC2.

According to one example, the indicator S3 determined in E6 (FIG. 8) comprises a scattering indicator (or score), i.e. an indicator representative of a level of scattering caused by the propagation of the ultrasonic waves in the medium during the ultrasonic scans SC1 and SC2. This scattering coefficient may define a scattering average pathway (or “mean free path” in English) which depends on the capacity of the insonified tissues to generate echoes resulting from multiple scattering phenomena.

More generally, the indicator S3 determined in E6 (FIG. 8) may be representative of any physiological, mechanical and/or tissue property, of the liver 3 and/or of the tissues 5 scrutinised by ultrasounds (elasticity, hardness, etc.) during the ultrasonic scans SC1 and SC2. The indicator S3 may comprise an indicator (or score) or a plurality of indicators (or scores).

According to one example, the indicator S3 determined in E6 (FIG. 8) comprises at least one from among the following indicators (or scores) as described before:
an indicator defining an attenuation coefficient;
an indicator defining a speed of sound;
an indicator defining a backscattering coefficient;
an indicator of the elasticity of the liver;
an indicator defining a viscoelasticity of the liver;
an indicator of propagation non-linearity; and
a scattering indicator.

According to one example, during the determination method (FIG. 8), the determination device 30 compares (E8) the indicator S3 obtained in E6 with a reference value RF1. This reference value RF1 may be any reference indicating a predetermined condition of a liver (health liver, particular hepatic disorder, etc.). For example, if the device 30 detects (E8) that the value of the indicator S3 obtained in E6 exceeds (or is lower than) the reference value, this means that the condition of the liver 3 of the subject UR1 is compliant (or is not compliant) with at least one predefined criterion.

According to one example, the indicator S3 defines the PDFF of the liver 3 of the subject UR1. During the comparison step E8, the value of the indicator S3 is compared with a threshold value RF1. If the obtained value of the PDFF exceeds the threshold value RF1 (or is within a range of predefined values), then a predefined condition of the liver 3 is detected. For example, the value of the PDFF may be interpreted as follows:
5.5% for an S1-grade steatosis or higher;
15.5% for an S2-grade steatosis or higher; and
20.5% for an S3-grade steatosis or higher.

As indicated for example in the document: Cassinotto, C., Jacq, T., Anselme, S., Ursic-Bedoya, J., Blanc, P., Faure, S., Belgour, A., & Guiu, B. (2022). Diagnostic Performance of Attenuation to Stage Liver Steatosis with MRI Proton Density Fat Fraction as Reference: A Prospective Comparison of Three US Machines. Radiology, 305(2), 353-361. https://doi.org/10.1148/radiol.212846.

It should be noted that, during the determination step E6 (FIG. 8), the indicator S3 may be determined in various manners by combining the scan data DT1a and DT1b resulting from the ultrasonic scans SC1 and SC2.

According to one example, the scan data DT1a and DT1b are data that are directly combined by the determination device 30 to deduce the indicator S3 therefrom. To do so, the determination method 30 applies, for example, a calculation function, denoted “f1”, on the scan data DT1a and DT1b so as to produce the indicator S3. In other words, the function f1 is applied so that: f1(DT1a; DT1b)=S3

According to another example, the determination device 30 carries out a first processing on the scan data DT1a and DT1b before determining the indicator S3. To do so, the device 30 applies, for example, functions f2 and f3 respectively on the scan data DT1a and DT1b so as to produce intermediate scores (or indicators) S1 and S2. In other words, the determination device 30 evaluates (or determines) a first score S1 (E4a, FIG. 8) from the first group of scan data DT1a and evaluates (or determines) a second score S2 (E4b, FIG. 8) from the second group of scan data DT1b. Afterwards, the determination device 30 may determine (E6) the score S3 from the first and second scores S1 and S2, for example by applying a function f4 on the intermediate scores S1 and S2.

The determination E6 of the indicator S3 then amounts to the following:
f2(DT1a)=S1;
f3(DT1b)=S2; and
f4(S1; S2)=S3.

It should be noted that the intermediate scores S1 and S2 could comprise at least one of any of the indicator types defined hereinbefore.

According to one example, the first score S1 is (or comprises) a first attenuation coefficient and the second score S2 is (or comprises) a second attenuation score. Thus, the intermediate scores S1 and S2 are representative of the attenuation of the ultrasonic waves propagating in the medium (the areas of interest) during the ultrasonic scans SC1 and SC2, respectively. In this case, the indicator S3 determined in E6 (FIG. 8) from the combination of the scan data DT1a and DT1b (and more specifically by combining the scores S1 and S2) may define, for example, a biomarker of a physiopathological condition of the liver.

The nature of the function f4 used by the device 30 to determine the indicator S3 from the intermediate scores S1 and S2 may be adapted as the case might be.

According to one example, the indicator S3 is determined from an average of the first and second scores S1 and S2. For example, this average is determined by a linear combination of the first and second scores S1 and S2. It may consist of a simple or weighted average. In other words, the indicator S3 may be calculated so that S3=aS1+bS2, wherein the weights a and b are set irrespective of the values of S1 and S2.

According to one example, the average used to determine S3 is a least mean-square average of S1 and S2 (also so-called LSM average, standing for “Least mean-square”).

According to one example, the average of S1 and S2 used to determine (E6, FIG. 8) the indicator S3 is a non-linear combination of the scores S1 and S2. In other words, the indicator S3 may be calculated so that S3=aS1+bS2, wherein the weights a and b are variable according to the values of S1 and S2. According to one example, one of the weights a or b may be zero for at least one value of the pair (S1, S2), so that only the intermediate score S1 or S2 having a non-zero weight is selected and used to determine S3.

According to one example, the non-linear combination of the scores S1 and S2 may be defined as f(S1, S2) or f(DT1a, DT1b), where f denotes a non-linear function.

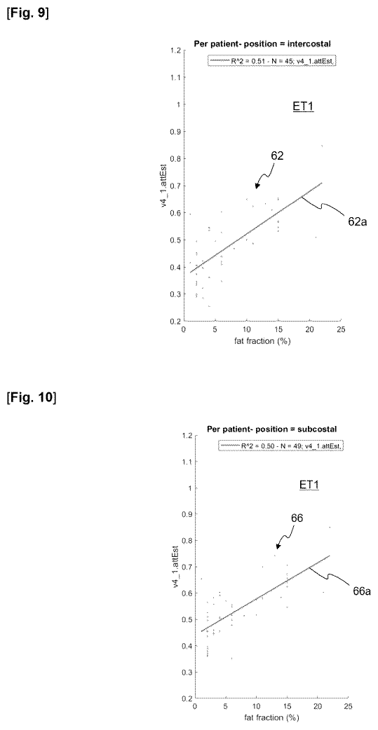

FIGS. 9 show, in the form of a cloud of points 62, the intermediate score S1 determined in E4a (FIG. 8) from the first scan data DT1a for the test subjects of a study denoted ET1 carried out by the Applicant. Similarly, FIGS. 10 shows, in the form of a cloud of points 66, the intermediate score S2 determined in E4b (FIG. 8) from the second scan data DT1b for the test subjects of the study ET1. In this example, the intermediate scores S1 and S2 define attenuation coefficients characterising the attenuation of the ultrasonic waves propagating in the subject UR1 during the first and second ultrasonic scans SC1 and SC2.

Linear regressions 62a and 66a have been determined, respectively, from the points 62 and 66 corresponding to the scores S1 and S2 resulting from the ultrasonic scans SC1 and SC2 (FIGS. 9 and 10). These linear regressions 62a and 66a represent the general evolution of the attenuation coefficients measured respectively in the intercostal and subcostal position as a function of the estimated PDFF score of each patient.

As it appears upon reading the study ET1, the performances reached in this example in the intercostal and subcostal ultrasonic scan configuration are close. The determination coefficients, denoted respectively r²_{CF1} and r²_{CF2}, have been determined based on the attenuation values (indicator S3) obtained in the intercostal and subcostal position (or mode). Thus, we obtain: r²_{CF1}=0.51 and r²_{CF2}=0.50. The correlation coefficient reflects the correlation between the PDFF score and the attenuation indicator. If this coefficient is equal to 1, then all the points are located on the same line. Thus, in this case, we obtain a correlation slightly higher in the subcostal configuration than in the intercostal configuration.

As a reminder, the determination coefficient r², also so-called Pearson linear determination coefficient, represents the quality of the prediction of a linear regression.

FIG. 11 shows, in the form of a cloud of points 70, the determined indicators S3 (E6) for the group of test subjects, evaluated during the study ET1. The indicators S3 define the attenuation indicator determined respectively for each subject of the study ET1, from the combination of the subcostal and intercostal attenuation coefficients of each subject of the group.

FIG. 12 shows, for reference, the distribution of the PDFF scores of the subjects of the study ET1 determined by a technique other than that of the determination method, namely by MRI imaging. The evaluation of the PDFF by MRI imaging is currently the reference for predicting or diagnosing a hepatic steatosis. It should be noted that this study ET1 covers, in this case, subjects having a PDFF lower than a limit value, namely 25%.

As shown in FIG. 11, a linear regression 70a has also been determined from the points 70 corresponding to the indicators S3 determined in E6 (FIG. 8) for each test subject of the study ET1. This linear regression 70a represents the general evolution of the overall attenuation coefficient as a function of the PDFF of the subjects. Advantageously, one could notice, in this example, a substantial improvement of the determination coefficient r² which amounts to 0.55 for the linear regression 70a. In other words, the linear regression 70a has a better correlation level of the overall attenuation coefficients S3 (in comparison with S1 and S2) with respect to the PDFF of the subjects, in comparison with the results obtained in the intercostal mode alone (FIG. 9) or in the subcostal mode alone (FIG. 10). This validates, for this example, the efficiency of the determination method which produces better results in terms of accuracy and reliability than the case where only one acquisition mode (intercostal or subcostal) was used.

According to an example illustrated in FIG. 8, once the indicator S3 is determined in E6, the determination device 30 evaluates (E10), from the indicator S3, a fat mass ratio, or PDFF, of the liver 3 of the subject UR1. Afterwards, the device 30 may possibly compare (E12) the obtained PDFF with a reference value RF2, this reference value being, for example, determined by an MRI imaging technique or another technology.

Moreover, the first scan data DT1a may be obtained before, or at the same time as, or after the second scan data DT1b. In particular, the first and second ultrasonic scans SC1 and SC2 may be carried out simultaneously or one after another, according to any order (SC1 before SC2 or SC2 before SC1), during the same scan session or different sessions.

According to one example, one amongst the first and second groups of scan data DT1a and DT1b is obtained from a scan, so-called anterior scan, carried out according to an anterior scan configuration and the other one is obtained from a scan, so-called posterior scan, carried out according to a posterior scan configuration, the anterior scan being carried out before the posterior scan. The anterior scan configuration is then one amongst the first and second scan configurations CF1 and CF2 and the posterior scan configuration is the other one amongst the first and second scan configurations CF1 and CF2.

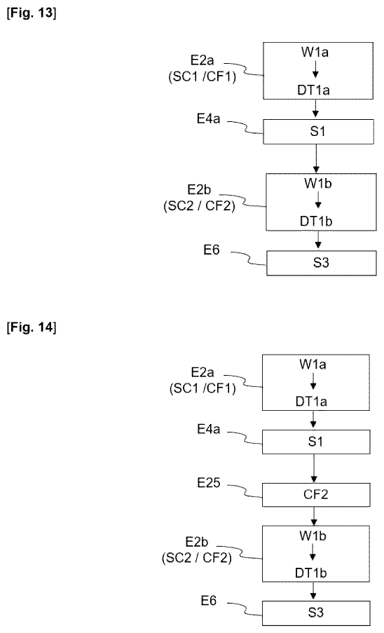

Thus, in a first example shown in FIG. 13, the anterior scan is the first ultrasonic scan SC1 carried out according to the anterior scan configuration (namely CF1) and the posterior scan is the second ultrasonic scan SC2 carried out according to the scan configuration (namely CF2). For example, the anterior scan configuration is the intercostal configuration and the posterior scan configuration is the subcostal configuration (the opposite being possible).

According to a second example, the anterior scan is the second ultrasonic scan SC2 carried out according to the anterior scan configuration (namely CF2) and the posterior scan is the first ultrasonic scan SC1 carried out according to the scan configuration (namely CF1).

Thus, it is possible to carry out the anterior scan, and then subsequently the posterior scan, for example during the same medical examination or during distinct medical examinations at different times (for example during a first examination and then a medical monitoring). In particular, it is possible to carry out the determination method of the disclosure in an incremental manner so that the determination device 30 generates a first result (for example S1 or S2) from the group of scan data resulting from the anterior scan, and then complete this result with the scan data resulting from the posterior scan in order to obtain the overall indicator S3. In this manner, it is advantageously possible to rapidly obtain a first result on the condition of the liver 3 of the subject UR1 and then improve this result subsequently thanks to the posterior scan.

According to an example shown in FIG. 14, the posterior scan configuration of the posterior scan (namely the configuration CF2 of the scan SC2 in this example) is selected (E25) from the first or second score S1 or S2 (namely S1 in this example) obtained from the anterior scan (namely SC1 in this example). Thus, it is advantageously possible to adapt the manner in which the posterior scan is carried out according to the result obtained before for the anterior scan. Depending on the result of the anterior scan, it might be wise to favour a particular scan configuration to best direct the ultrasonic observation.

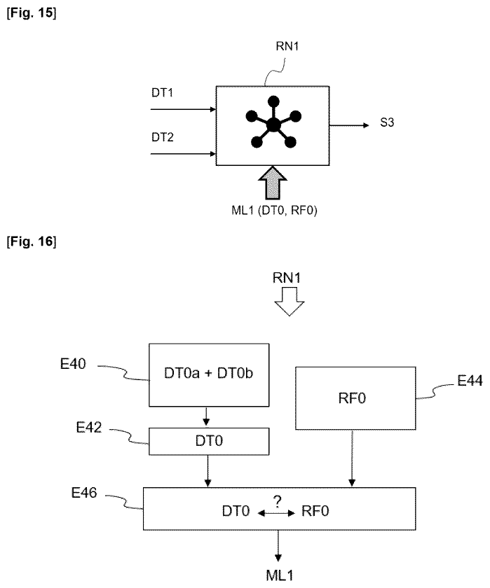

According to an example shown in FIG. 15, the determination device 30 (FIG. 3) carries out, during the determination step E6 (FIG. 8), a processing of the first and second groups of scan data DT1a and DT1b, or data obtained from these data DT1a and DT1b, to determine the overall indicator S3. This processing is carried out according to a processing model ML1 obtained using a (or at least one) neural network RN1 trained from training data DT0 and reference scores (or indicators, or values) RF0. To do so, the neural network RN1 may execute an algorithm based on an artificial intelligence module. The training data with which the neural network RN1 is trained may comprise ultrasonic scan data or data obtained from such ultrasonic scan data.

According to an example shown in FIG. 16, prior to the aforementioned processing, the neural network RN1 is trained by machine learning during a training phase E40-E46. During this training phase, the neural network RN1 obtains (E40) ultrasonic scan data DT0a and DT0b representative of waves propagating respectively according to the first and second scan configurations CF1 and CF2 on test subjects. Afterwards, the neural network RN1 determines (E42) training data DT0 from the ultrasonic scan data DT0a and DT0b. These training data DT0 may comprise the scan data DT0a and/or DT0b themselves of data obtained from these data DT0a and/or DT0b. The neural network RN1 further obtains (E44), from MRI imagings carried out on the test subjects, reference scores RF0 representative of the condition of the liver of the test subjects. Thus, the neural network RN1 may determine (E46), by comparison of the training data DT0 with the reference scores RF0, the processing model ML1 used to estimate the indicator S3 from the combination of the first and second groups of scan data DT1a and DT1b.

According to one example, during the determination step E6 (FIG. 8), the determination device 30 successively executes a first algorithm and then a second algorithm to evaluate the condition of the liver 3 of the subject UR1. The first algorithm is an estimation algorithm configured to determine an overall attenuation coefficient S3 from the first and second groups of scan data DT1a and DT1b obtained in the intercostal and subcostal mode, respectively. For example, this first estimation is done by averaging the attenuation coefficients S1 and S2 obtained intercostally or subcostally, as already described. The second algorithm is executed by a neural network (such as the network RN1) to estimate the PDFF of the liver 3 of the subject UR1 from the overall attenuation coefficient S3, for example from the model ML1 or from a linear regression as described before. Thus, the statistical model used in the second algorithm may correlate the overall indicator S3 (representing for example at least one of the aforementioned parameters: attenuation coefficients, speed of sound, etc.) and an estimation of the PDFF at the output.

According to one example, the determination device 30 may implement a neural network RN1 to generate a model ML1 correlating ultrasonic images acquired at the input from the ultrasonic scans SC1 and SC2, on the one hand, and the indicator S3 or the PDFF obtained at the output.

Advantageously, the determination method and device of the present disclosure allow determining, reliably and effectively, the condition of the liver of a subject who could be healthy or have any physiopathological condition of the liver. To do so, the concept of the present disclosure is based on the combination of scan data (or ultrasonic data) obtained according to two distinct scan configurations, namely according to respectively a first scan configuration CF1 targeting the liver and a second scan configuration CF2 targeting the liver or another group of tissues of the subject. Advantageously, the combination of these scan data allows obtaining an indicator S3 representing the condition of the liver of the subject in a more robust manner than the case where only one scan configuration was used.

The combination of two distinct acquisition modes, for example in the intercostal and subcostal position (or mode), advantageously allows improving the quality of the acquisition data (with data of different kinds) in comparison with a conventional technique where only ultrasonic data obtained in the intercostal mode would have been taken into account. The ultrasonic data thus acquired are less correlated with one another. Advantageously, using two distinct scan configurations allows diversifying the measurements and thus enhancing the reliability and the accuracy of the results.

Furthermore, in the case of hepatic steatosis for example, all the regions of the liver of the subject could be affected in different manners. Thus, some cases of non-uniform steatoses have been noticed. The multiplication of the ultrasonic scans according to different configurations allows obtaining a more representative estimate of the overall condition of the liver of the subject.

Thus, the present disclosure may advantageously provide assistance in the diagnosis or in the monitoring of some physiopathological conditions of the liver, such as hepatic steatosis (for example of the NASH type), steatohepatitis, etc.

As a person skilled in the art understands, all of the embodiments and variants described above, some of which have been deliberately simplified to make them easier to explain, only constitute non-limiting exemplary embodiments of the present invention. In particular, a person skilled in the art could envisage adapting or combining the embodiments and variants described above in order to address a specific need.

Hence, the present disclosure is not limited to the previously-described embodiments but covers in particular a steering method that would include secondary steps without departing from the scope of the present disclosure. The same would apply to a steering system for the implementation of such a method.

1. A method for determining the condition of the liver (3) of a subject (UR1), said method comprising:
obtaining (E2a) a first group of scan data (DT1a) from ultrasonic waves propagating according to a first scan configuration (CF1) of the liver;
obtaining (E2b) a second group of scan data (DT1b) from ultrasonic waves propagating according to a second scan configuration (CF2) of a group of tissues (5) of the subject; and
determining (E6), by a combination of the first and second groups of scan data, an indicator S3 representative of the condition of the liver (3).

2. The method according to claim 1, wherein:
the group of tissues (5) of the subject corresponds to a portion of the subject other than the liver (3).

3. The method according to claim 1, wherein:
the first scan configuration (CF1) is an intercostal scan configuration of the liver; and
the second scan configuration (CF2) is a subcostal scan configuration of the liver.

4. The method according to any one of the preceding claims, wherein the indicator S3 comprises at least one from among:
an indicator defining an attenuation coefficient;
an indicator defining a speed of sound;
an indicator defining a backscattering coefficient;
an indicator of the elasticity of the liver;
an indicator defining a viscoelasticity of the liver; and
an indicator of propagation non-linearity.

5. The method according to any one of the preceding claims, comprising:
comparing the indicator S3 with a reference value (RF1).

6. The method according to any one of the preceding claims, wherein said method comprises:
evaluating (E4a) a first score (S1) from the first group of scan data (DT1a); and
evaluating (E4b) of a second score (S2) from the second group of scan data (DT1b);
wherein the indicator S3 is determined from the first and second scores.

7. The method according to claim 6, wherein:
the first score (S1) is a first attenuation coefficient; and
the second score (S2) is a second attenuation coefficient;
wherein the indicator S3 defines a biomarker of a physiopathological condition of the liver.

8. The method according to claim 7, wherein one amongst the first and second groups of scan data (DT1a, DT1b) is obtained from a scan so-called anterior scan according to an anterior scan configuration and the other one is obtained from a scan so-called posterior scan carried out according to a posterior scan configuration, the anterior scan being carried out before the posterior scan;
wherein the anterior scan configuration is one amongst the first and second scan configurations (CF1, CF2) and the posterior scan configuration is the other one amongst the first and second scan configurations.

9. The method according to claim 8, wherein the posterior scan configuration is selected from among the first or second score (S1, S2) obtained from the anterior scan.

10. The method according to any one of claims 6 to 9, wherein the indicator S3 is determined from an average of the first and second scores (S1, S2).

11. The method according to claim 10, wherein the average is determined by a linear combination of the first and second scores (S1, S2).

12. The method according to claim 11, wherein the average is a least mean-square average.

13. The method according to claim 10, wherein the average is determined by a non-linear combination of the first and second scores (S1, S2).

14. The method according to claim 13, wherein the determination of the indicator S3 comprises:
-processing the first and second groups of data (DT1a, DT1b), or data obtained from the first and second groups of data, according to a processing model obtained using a neural network (RN1) trained from training data (DT0) and reference scores (RF0), the training data comprising ultrasonic scan data or data obtained from the ultrasonic scan data.

15. The method according to claim 14, wherein, prior to the processing, the neural network (RN1) is trained by machine learning during a training phase comprising:
obtaining (E40) the ultrasonic scan data representative of waves propagating according to the first and second scan configurations on test subjects;
determining (E42) the training data from the ultrasonic scan data;
obtaining (E44), from MRI imagings carried out on the test subjects, the reference scores representative of the condition of the liver of the test subjects; and
determining (E46), by comparison of the training data with the reference scores, the processing model (ML1) used to estimate the indicator S3 from the combination of the first and second groups of scan data.

16. The method according to any one of the preceding claims, wherein the method comprises:
evaluating, from the indicator S3, a fat mass ratio of the liver of the subject.

17. A computer program (PG1) including instructions for the implementation of the method according to any one of the preceding claims, when these instructions are executed by a processor.

18. A device (30) for determining the condition of the liver (3) of a subject (UR1), said device comprising:
a first obtaining module (MD2) configured to obtain a first group of scan data from ultrasonic waves propagating according to a first scan configuration of the liver;
a second obtaining module (MD4) configured to obtain a second group of scan data from ultrasonic waves propagating according to a second scan configuration of a group of tissues of the subject; and
an analysis module (MD6) configured to determine, by a combination of the first and second groups of scan data, an indicator S3 representative of the condition of the liver.

19. A system for determining (SY2) the condition of the liver of a subject comprising:
the determination device (30) according to claim 18; and
an ultrasonic system (SY1) coupled to the determination device.
